(19) United States
(12) Patent Application Publication
(10) Pub. No.: US 2007/0208243 A1
(43) Pub. Date: Sep. 6, 2007

(54) Nanoelectronic glucose sensors

(75) Inventors: Jean-Christophe P. Gabriel, Pinole, CA (US); Shripal Gandhi, Emeryville, CA (US); Alexander Star, Pittsburgh, PA (US); Christian Valcke, Orinda, CA (US)

Correspondence Address: O'MELVENY & MYERS LLP, 610 NEWPORT CENTER DRIVE, 17TH FLOOR, NEWPORT BEACH, CA, 92660, US

(73) Assignee: Nanomix, Inc., Emeryville, CA (US)

(21) Appl. No.: 11/274,747

(22) Filed: Nov. 15, 2005

Related U.S. Application Data
(63) Continuation-in-part of application No. 10/945,803, filed on Sep. 20, 2004.
(63) Continuation-in-part of application No. 10/846,072, filed on May 14, 2004.
(63) Continuation-in-part of application No. 10/773,631, filed on Feb. 6, 2004.
(63) Continuation-in-part of application No. 10/704,066, filed on Nov. 7, 2003.
(63) Continuation-in-part of application No. 10/656,898, filed on Sep. 5, 2003.
(63) Continuation-in-part of application No. 10/345,783, filed on Jan. 16, 2003.
(60) Provisional application No. 60/471,243, filed on May 16, 2003.
(60) Provisional application No. 60/504,663, filed on Sep. 18, 2003.
(60) Provisional application No. 60/445,654, filed on Feb. 6, 2003.
(60) Provisional application No. 60/424,892, filed on Nov. 8, 2002.
(60) Provisional application No. 60/408,547, filed on Sep. 5, 2002.
(60) Provisional application No. 60/349,670, filed on Jan. 16, 2002.

(51) Int. Cl. A61B 5/05

(52) U.S. Cl. 600/347; 600/345

(57) ABSTRACT

A nanostructured electronic device for detection and measurement of biomolecules, such as blood glucose. Also disclosed are methods of using and manufacturing devices employing nanotubes as electronic transducers.

### CLAIMS OF PRIORITY TO PRIOR PATENT APPLICATIONS

This application claims priority pursuant to 35 USC Section 119(e) to each of the following U.S. Provisional Applications: No. 60/627,743 filed Nov. 13, 2004 entitled “Nanotube Based Glucose Sensing”; and No. 60/723,530 filed Oct. 3, 2005 entitled “Sensor Array based on metal decorated carbon nanotubes”.

This application also is a continuation-in-part of and claims priority to U.S. patent application Ser. No. 10/945,803 filed Sep. 20, 2004 entitled “Multiple Nanoparticles Electrodeposited On Nanostructures”, which in turn claims priority to U.S. Provisional Patent Application No. 60/504,663 filed Sep. 18, 2003.

This application is also a continuation-in-part of co-pending U.S. patent application Ser. No. 10/846,072 filed May 14, 2004 entitled “Flexible nanotube transistors”, which claims priority to U.S. Provisional Patent Application No. 60/471,243 filed May 16, 2003.

This application is also a continuation-in-part of co-pending U.S. patent application Ser. No. 10/773,631 filed Feb. 6, 2004 entitled “Analyte Detection In Liquids With Carbon Nanotube Field Effect Transmission Devices”, which claims priority to U.S. Provisional Patent Application No. 60/445,654 filed Feb. 6, 2003.

This application is also a continuation-in-part of co-pending U.S. patent application Ser. No. 10/704,066 filed Nov. 7, 2003 entitled “Nanotube-Based Electronic Detection Of Biomolecules” (published 2004-0132,070), and likewise claims the full priority chain of Ser. No. 10/704,066, which is described as follows:
a) application Ser. No. 10/704,066 claims priority to U.S. Provisional Application No. 60/424,892 filed Nov. 8, 2002 entitled “Nanotube-based electronic detection of biomolecules”;
b) application Ser. No. 10/704,066 is a continuation-in-part of two co-pending US Applications:
   (i) Ser. No. 10/345,783 filed Jan. 16, 2003 entitled “Electronic Sensing Of Biological And Chemical Agents Using Functionalized Nanostructures”, which claims priority to Provisional Application No. 60/349,670 filed Jan. 16, 2002; and
   (ii) Ser. No. 10/656,898 filed Sep. 5, 2003 entitled “Polymer Recognition Layers For Nanostructure Sensor Devices”, which claims priority to Provisional Application No. 60/408,547 filed Sep. 5, 2002.

Each of the foregoing provisional and non-provisional applications is specifically incorporated herein, in its entirety, by reference.

### CROSS-REFERENCE TO RELATED APPLICATIONS

This application is related to the subject matter of the following patent applications by Joel S. Douglas:
a) U.S. patent application Ser. No. 11/063,504, filed Feb. 23, 2005 (Published 2005-0186,333) entitled “Strip Electrode With Conductive Nano Tube Printing”. This application is believed to claim the priority of:
   (i) U.S. Provisional Application No. 60/546,762 filed Feb. 23, 2004, entitled “Strip Electrode With Conductive Nano Tube Printing”; and
   (ii) U.S. Provisional Application No. 60/547,665 filed Feb. 25, 2004, entitled “Strip Electrode With Conductive Nano Tube Printing and Methods”.
b) International Patent Application No. PCT/US05/06,131 filed on Feb. 23, 2005, which is believed to claim the priority of above identified '762 and '665 Provisional Applications.

Each of the above identified related patent applications is specifically incorporated herein, in its entirety, by this reference.

### BACKGROUND OF THE INVENTION

1. Field of the Invention

The present invention relates to sensors for chemical species using nanostructured electronic devices, and methods relating to their use and manufacture, and in particular, to devices employing nanotubes as electronic transducers for the detection and measurement of solvated biomolecules or physiologic species, such as blood glucose.

2. Description of Related Art

A significant percentage of the US population suffers from diabetes (18.2 million or 6.3%). Of the 18.2 million, 13 million have been diagnosed and of the diagnosed fraction 4 million people take insulin daily. This patient group is supported through numerous foundations and professional associations who provide patient care and education.

Sources report the market growth at 15% annually, driven mainly by increased incidence of disease (obesity, diet) and increased daily monitoring by present patients. Insulin is taken to regulate blood glucose level. The amount of insulin taken must be titrated based on food intake, exercise, physical condition of the user plus the current level of glucose. For the 4 million who follow the insulin dosing protocol blood glucose measurements are suggested 4 to 6 times per day. Diabetics who are not insulin dependent check their blood glucose less frequently, typically 1 or two times a day, to adjust oral medications as well as exercise and food intake. It is estimated that this results in about 9-10 billion glucose determinations per year worldwide.

Self-measurement of glucose is common. Measuring one's own glucose level is typically called Self Monitoring of Blood Glucose (SMBG). Most SMBG readings are done on a sample of capillary blood obtained by a finger prick. The blood is applied to a disposable sensor “strip” typically an electrochemical sensor containing Glucose Oxidase (GOX). The sensor current or voltage is read by a small electrometer referred to as a glucose meter.

An example of a popular glucose meter is shown in FIG. 1A. The strips are contained in a cartidge. The meter automatically pushes the strips out to collect blood. After use the user must manually remove each strip and dispose of it. Most glucose meters are battery driven and have a measurement range of 20-600 mg/dL. Required blood volume varies between 0.3 and 1 uL. Most meters are provided freely to get payback on strip usage. Disposable strips contain the actual glucose sensor. Capillary action is used to move the blood into the area of the sensor.

FIG. 1B shows a wearable glucose sensor. However, there is a need for glucose measurement and monitoring technology which is more convenient, cheaper, and better suited to integration into other systems, such as invasive or implantable diagnostic or therapeutic devices.

### SUMMARY OF THE INVENTION

It should be understood that one aspect of the invention herein may be set forth in one part of the description, figures, formulas, and/or examples herein, while other aspects of the invention may be set forth in other parts of the description, figures, formulas, and/or examples herein. Certain advantageous inventive combinations may be taught in one part of the description, figures, formulas, and/or examples herein, and the detailed description, and the best mode of such combinations and their respective elements may be set forth in other parts of the description, figures, formulas, and/or examples herein. Therefore the invention is to be understood broadly from this disclosure as read in its entirety, including the patent applications incorporated by reference, and including the informal claims set forth below.

Certain embodiments of a nanoelectronic patient medical monitor system having aspects of the invention comprise, an electronic control processor disposed in a patient-portable housing; an analyte fluid sampling device, comprising at least one micro-needle disposed in the patient-portable housing, and configured to draw a sample of body fluid; a plurality of nanosensors disposed in the a patient-portable housing and in fluid communication with the analyte fluid sampling device; each of the plurality of nanosensors including: a substrate; a nanostructured layer comprising a plurality of nanostructures disposed adjacent the substrate; at least one electrical contact disposed adjacent the substrate in electrical communication with the nanostructured layer; the nanostructured layer configured to transmit at least one signal via the at least one contact in response to a target analyte; electrical measurement circuitry disposed in a patient-portable housing in communication with the electronic control processor and in selectable communication with the at least one electrical contact of each nanosensor, the measurement circuitry configured to detect a concentration of a target analyte using the at least one signal; a plurality of electronically actuated valves disposed in association with the analyte fluid sampling device and in communication with the electronic control processor, each one of the plurality of electronically actuated valves disposed adjacent a respective one of a plurality of nanosensors and configured to regulate the fluid communication of the analyte fluid sampling device with a respective nanosensor; and the electronic control processor further including a memory and code instructions configured to selectably actuate one or more of the electronically actuated valves to provide body fluid sample to one or more selected nanosensors, and to cause the electrical measurement circuitry to detect a concentration of the target analyte using the at least one signal from the respective nanosensor.

Certain exemplary embodiments having aspects of the invention comprise an electronic sensor device configured preferably as a wearable monitor, which provides the convenience of longer term monitoring (e.g., 1 week), optionally with a disposable sensor element which provides cost effective benefits to patients.

In certain embodiments, it is advantageous to make each sensor a single-use device, the sensor being integrated into a reusable measurement system. Alternative embodiments may include an array with multiple sensor elements on a chip, wherein the multiple sensors are configured to be used sequentially by the patient or care provider, so that the device can provide a plurality of measurements. These embodiments are arranged to take advantage of the photolithographic manufacturing technology common in the electronics industry to reduce the cost-per-measurement to a low level. Known microprocessors, output devices, displays and/or power sources and the like may be included in the sensor system.

Additional exemplary embodiments having aspects of the invention comprise an electronic sensor device which is biocompatible and configured to be operated with all or a portion of the device emplaced or inserted within a patient's body. Known biocompatible materials may be readily used to construct the sensor device.

In certain embodiments, one or more sensor devices are integrated into or coupled to a drug delivery system, such as an implantable insulin delivery device. The electronic sensor device is configured so as to control the release of one or more drugs in relation to the measured blood concentration of one or more target species, such as the controlled release of insulin relative to monitored blood glucose level.

Optionally, the nanoelectronic patient medical monitor system may further comprise an electronically controllable drug delivery system in communication with the electronic control processor, configured to deliver a selected dosage of a medication in response to the detect of a target analyte in the body fluid sample.

Additional exemplary embodiments having aspects of the invention comprise an insulating substrate such as a polymeric base film or strip, further comprising printed or deposited conductive material configured as at least one electrode region on or adjacent the substrate surface, and further comprising a coating in communication with at least a portion of the electrode regions, the coating including at least a functionalized nanostructure such as a film of carbon nanotubes functionalized with a metal and/or an organic material. The functionalization may include a bio-selective material such as a glucose-reactive enzyme.

Certain sensor device embodiments having aspects of the invention comprise a substrate having a conductive layer, the conductive layer comprising a plurality of nanostructures (e.g., SWNTs, MWNTs, nanowires and other nanoparticles of various compositions), and preferably a network or film of single-walled carbon nanotubes. The conductive layer preferably has functionalization material or reacted groups, which may include a quantity of platinum (Pt) nanoparticles, preferably deposited on or bound to the nanostructures, such as SWNTs. In a preferred embodiment, Pt nanoparticles are produced and bound to SWNTs in solution or dispersion phase by reduction of a soluble Pt compound in a suitable solvent, the Pt functionalized SWNTs then being printed, sprayed or otherwise deposited on the substrate to form the conductive layer. Preferably a detection enzyme, such as glucose oxidase (GOx) is disposed on or in association with the conductive layer. The sensor device may include a counter electrode disposed adjacent the conductive layer in a spaced-apart fashion, such as on a second substrate arranged adjacent the first substrate, the space between the counter electrode and conductive layer forming a sample cell for an analyte medium, for example, blood (suitable containing elements may be included to immobilize the analyte medium during sensor operation). Both the counter electrode and the conductive layer may be connected to suitable measurement circuitry to detect a change in an electrical property of the sensor in response to the presence of a target analyte. For example, glucose in a blood sample may react with GOx to form reaction products, such as hydrogen peroxide (H₂O₂) and gluconic acid), which in turn electrochemically generate a current flow between the counter electrode and the conductive layer, which can be measured as an indication of glucose concentration.

Certain alternative sensor device embodiments having aspects of the invention comprising a conductive layer having nanostructures functionalized by binding to a conductive polymeric material, for example a polyaniline derivative such as poly (m-aminobenzene sulfonic acid) or PABS. This composite material may be employed with or without Pt nanoparticles. Certain alternative sensor architectures may be employed in association with a conductive layer comprising a nanostructure/conductive polymer composite (such as SWNT/PABS) for detection of analytes, without departing from the spirit of the inventions. For example the sensor may be configured as a resistance sensor, an FET, a capacitance or impedance sensor, or the like, and may be arranged as an array including a combination of these.

### BRIEF DESCRIPTION OF THE DRAWINGS

The figures and drawings may be summarized as follows:

FIG. 1A shows an existing glucose sensor;

FIG. 1B shows an existing wearable glucose sensor;

FIG. 2A is a schematic cross section view of a nanostructure device having aspects of the invention with a recognition layer specific to a selected analyte.

FIG. 2B is a schematic diagram of GOx functionalization of a nanotube sensor embodiment;

FIG. 3 is a plot showing the response of a sensor embodiment to glucose in water.

FIG. 4 is a schematic cross section view of an alternative sensor having aspects of the invention.

FIGS. 5A and 5B show examples of available sampling needles;

FIG. 6 is a schematic cross section view of a multi-well sensor system having electronically controlled sample ports.

FIG. 7 (Views A-C) illustrate an exemplary electrochemical sensor configured as a blood test strip.

FIG. 8 is a plot showing the response of a electrochemical sensor embodiment functionalized with SWNT-PABS.

### DETAILED DESCRIPTION OF THE EMBODIMENTS

The making, functionalization and use of nanostructured chemosensor and biosensor devices is described in considerable detail in the patent applications incorporated by reference above.

### EXAMPLE A

### A Nanostructured Glucose Sensor Device

Sensor Platform Architecture.

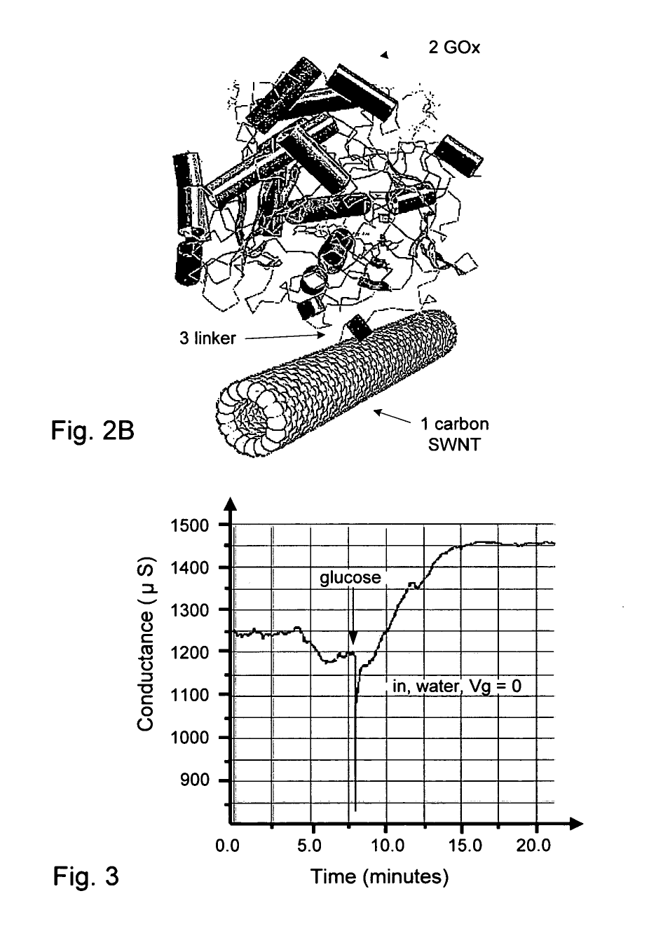

A number of preferred sensor embodiments employ nanostructures, such as nanotubes. FIG. 2A. shows an electronic sensing device 10 for detecting a liquid or gaseous analyte 11, comprising a nanostructure sensor 12. Device 12 comprises a substrate 1, and a conducting channel or layer 2 including nanostructured material disposed upon a substrate 1.

The nanostructured material may contact the substrate as shown, or in the alternative, may be spaced a distance away from the substrate, with or without a layer of intervening material. As used herein, a “nanostructured material” includes any object or objects which has at least one dimension smaller than about 100 nm and comprises at least one sheet of crystalline material with graphite-like chemical bonds. Examples include, but are not limited to, single-walled nanotubes, double-walled nanotubes, multi-walled nanotubes, and “nanoonions.” Chemical constituents of the crystalline material include, but are not limited to, carbon, boron nitride, molybdenum disulfide, and tungsten disulfide. Preferably a nanotube is a carbon nanotube, and more preferably it is a single-walled carbon nanotube.

In an embodiment of the invention, conducting channel 2 may comprise one or more carbon nanotubes. For example, conducting channel 2 may comprise a plurality of nanotubes forming an interconnecting mesh, film or network Typically, a “nanotube network” is a film of nanotubes disposed on a substrate in a defined area. A film of nanotubes comprises at least one nanotube disposed on a substrate in such a way that the nanotube is substantially parallel to the substrate. The film may comprise many nanotubes oriented generally parallel to each other. Alternatively, the film may comprise many nanotubes, each oriented substantially randomly with respect to adjacent nanotubes, or the nanotubes may be oriented substantially perpendicular to the substrate, e.g., as in a “nano-turf” configuration, or combinations of the foregoing.

The number of nanotubes in an area of substrate is referred to as the density of a network. Preferably, the film comprises many nanotubes oriented substantially randomly, with the density high enough that electric current may pass through the network from one side of the defined area to the other side. Methods for disposing a high density of nanotubes are disclosed in U.S. patent application Ser. No. 10/177,929, filed Jun. 21, 2002 by Gabriel et al., which is herein incorporated by reference, in its entirety.

Substrates may be flat objects that are electrically insulating. Substrates have a chemical composition, of which examples include, but are not limited to, silicon oxide, silicon nitride, aluminum oxide, polyimide, and polycarbonate. Preferably the substrate is a silicon oxide film on the silicon chip.

One or more conductive elements or contacts 3a, 3b may be disposed over the substrate and electrically connected to conducting channel 2. Elements 3a, 3b may comprise metal electrodes in direct contact with conducting channel 2. In the alternative, a conductive or semi-conducting material (not shown) may be interposed between contacts 3a, 3b and conducting channel 2. Contacts 3a, 3b may comprise a source electrode S and a drain electrode D upon application of a selected and/or controllable source-drain voltage Vsd (note that the voltage and/or polarity of source relative to drain may be variable, e.g., current may be DC, AC and/or pulsed, and the like). In such case, the contacts are arranged so that the nanotube network comprises at least one conductive path between at least a pair of conductors.

Alternatively, a contact or electrode may be employed to provide a charge to the channel 2 relative to a second electrode, such that there is an electrical capacitance between the second electrode and the channel 2. The second electrode may be a gate electrode, a discrete bottom electrode (e.g. embedded in, under, and/or doped within the substrate), a top gate electrode, a liquid medium electrode, and the like. In another exemplary preferred embodiment, the gate electrode is a conducting element in contact with a conducting liquid, said liquid being in contact with the nanotube network. In other embodiments, the device includes a counter electrode, reference electrode and/or pseudo-reference electrode.

In one exemplary preferred embodiment, the gate electrode is a conducting plane within the substrate beneath the silicon oxide. Examples of such nanotube electronic devices are provided, among other places, in patent application Ser. No. 10/656,898, filed Sep. 5, 2003 and Ser. No. 10/704,066, filed Nov. 7, 2003, both of which are incorporated herein, in their entirety, by reference. FIG. 2A, the device 10 may operate as a gate-controlled field effect transistor via the effect of gate electrode 4. In this example, the gate 4 comprises a base portion of the substrate, such as doped-silicon wafer material isolated from contacts 3a, 3b and channel 2 by dielectric 5, so as to permit a capacitance to be created by an applied gate voltage Vg. For example, the substrate 1 may comprise a silicon back gate 4, isolated by a dielectric layer 5 of SiO2. Such devices are generally referred to herein as nanotube field effect transistors (NTFET).

Embodiments of an electronic sensor device having aspects of the invention may include an electrical circuit configured to measure one or more properties of the nanotube sensor, such as measuring an electrical property via the conducting elements. For example, a conventional power source may supply a source drain voltage Vsd between contacts 3a, 3b. Measurements via the sensor device 10 may be carried out by circuitry represented schematically by meter 6 connected between contacts 3a, 3b. In embodiments including a gate electrode 4, a conventional power source may be connected to provide a selected and/or controllable gate voltage Vg. Device 10 may include one or more electrical supplies and/or a signal control and processing unit (not shown) as known in the art, in communication with the sensor 12.

Any suitable electrical property may provide the basis for sensor sensitivity, for example, electrical resistance, electrical conductance, current, voltage, capacitance, transistor on current, transistor off current, and/or transistor threshold voltage. Alternatively, sensitivity may be based on measurements including a combination, relationship, pattern and/or ratios of properties and/or the variation of one or more properties over time. For example, the capacitance or impedance of the nanostructures relative to a gate or counter electrode. Similarly, a breakdown voltage or electron emission voltage and/or current may be measured between nanostructures and a reference electrode.

In certain embodiments, a transistor sensor may be controllably scanned through a selected range of gate voltages, the voltages compared to corresponding measured sensor current flow (generally referred to herein as an I-Vg curve or scan). Such an I-Vg scan may be through any selected gate voltage range and at one or more selected source-drain potentials. The Vg range is typically selected from at least device “on” voltage through at least the device “off” voltage. The scan can be either with increasing Vg, decreasing Vg, or both, and may be cycled ± at any selected frequency.

From such measurements, and from derived properties such as hysteresis, time constants, phase shifts, and/or scan rate/frequency dependence, and the like, correlations may be determined with target detection and/or concentration and the like. The electronic sensor device may include and/or be coupled with a suitable microprocessor or other computer device of known design, which may be suitably programmed to carry out the measurement methods and analyze the resultant signals. Those skilled in the art will appreciate that other electrical and/or magnetic properties, and the like may also be measured as a basis for sensitivity. Accordingly, this list is not meant to be restrictive of the types of device properties that can be measured.

In certain embodiments, sensor 12 may further comprise a layer of inhibiting or passivation material 6 covering regions adjacent to the connections between the conductive elements 3a, 3b and conducting channel 2. The inhibiting material may be impermeable to at least one chemical species, such as the analyte 11. The inhibiting material may comprise a passivation material as known in the art, such as silicon dioxide, aluminum oxide, silicon nitride, and the like. Further details concerning the use of inhibiting materials in a NTFET are described in prior application Ser. No. 10/280,265, filed Oct. 26, 2002, entitled “Sensitivity Control For Nanotube Sensors” (published as US 2004-0043527 on Mar. 4, 2004) which is incorporated by reference herein.

The conducting channel 2 (e.g., a carbon nanotube layer) is typically functionalized to produce a sensitivity to one or more target analytes 11. Although nanoparticles such as carbon nanotubes may respond to a target analyte through charge transfer or other interaction between the device and the analyte, more generally a specific sensitivity can be achieved by employing recognition material 7 that induces a measurable change the device characteristics upon interaction with a target analyte. Typically, the sensor functionalization layer 7 is selected for a specific application. The analyte may produce the measurable change by electron transfer, and/or may influence local environment properties, such as pH and the like, so as to indirectly change device characteristics. Alternatively or additionally, the recognition material may induce electrically-measurable mechanical stresses or shape changes in the conducting channel 2 upon interaction with a target analyte.

In a typical embodiment having aspects of the invention, the sensitivity is produced and/or regulated by the association of the nanotube layer 2 with a functionalization material, e.g. disposed as a functionalization layer 7 adjacent channel 2. The functionalization layer 7 may be of a composition selected to provide a desired sensitivity to one or more target species or analytes. The functionalization material may be disposed on one or more discrete portions of the device, such as on all or a portion of the channel 2, or alternatively may be dispersed over the sensor 12, such as on contacts 3 and/or exposed substrate 1.

Optionally device 10 may comprise a plurality of sensors 12 disposed in a pattern or array, as described in U.S. patent application Ser. No. 10/388,701 filed Mar. 14, 2003 entitled “Modification Of Selectivity For Sensing For Nanostructure Device Arrays” (now published as US 2003-0175161). Each device in the array can be functionalized with identical or different functionalization. Identical device in an array can be useful in order to multiplex the measurement to improve the signal/noise ratio or increase the robustness of the device by making redundancy.

Glucose Sensor Functionalization.

The above described sensor embodiments, such as a preferred embodiment of a carbon nanotube network transistor, may be treated or engaged with many alternative functionalization materials, probes, molecular transducers, coatings and the like.

In one example of a glucose sensor, the network is functionalized using the enzyme glucose oxidase (GOx), so as to provide glucose-specific sensitivity. FIG. 2B diagrammatically illustrates the functionalization. In one preferred example, the GOx (or an alternative biomolecule probe) may be bonded to a linker molecule, such as pyrene, polymer or the like. (See patent application Ser. No. 10/345,783 incorporated by reference above).

The linker molecule, in turn, is selected to have properties which cause it to associate with the lattice of the carbon nanotube, such as by non-covalent pi-pi stacking between the graphitic nanotube lattice and the flat ring pyrene structure. (see Besteman, et al., Enzyme Coated Carbon Nanotubes As Single Molecule Biosensors, Nano Letters, 2003 Vol. 3, No. 6, 727-730) Such a functionalization structure is referred to as a molecular transducer.

In operation of the sensor, the immobilized GOx reacts with glucose presented in the contacting medium so as to alter the electrical properties of the nanotube device. FIG. 3 shows a plot of the response of a NT sensor device (in this example the device is a NTFET with Vgate=0). Initially, in a water medium without glucose, the conductance is about 1200-1250 muS. Upon injection of a glucose sample (at arrow) the conductance rises to a stable level of about 1450 muS. There is an very brief transient at injection, which is believed to be an artifact of the injection process (brief exposure to air). The change in conductance may be correlated with the concentration of glucose. The response time and transient may be controlled by appropriate sample presentation, such as by rapid mixing to equilibrium concentration in contact with functionalized nanotube network.

In operation of the sensor, the immobilized GOx reacts with glucose presented in the contacting medium so as to alter the electrical properties of the nanotube device. FIG. 3 shows a plot of the response of a NT sensor device (in this example the device is a NTFET>generally as shown in FIG. 2A) with Vgate=0). Initially, in a water medium without glucose, the conductance is about 1200-1250 μS. Upon injection of a glucose sample (at arrow) the conductance rises to a stable level of about 1450 μS. There is an very brief transient at injection, which is believed to be an artifact of the injection process (brief exposure to air). The change in conductance may be correlated with the concentration of glucose. The response time and transient may be controlled by appropriate sample presentation, such as by rapid mixing to equilibrium concentration in contact with functionalized nanotube network. The response of the NT sensor toward an increased conductance is indicative of the effect of the biochemical environment (GOx reacting with aqueous glucose solution) on the conductance of the nanotube network channel 2 as measured between source 3a and drain 3b.

Alternative Functionalizations

Alternative enzymes may be employed for chemodetection and biodetection in a manner similar to that described above. For example, an alternative glucose sensor system embodiment is functionalized using the enzyme methane reductase, so as to provide sensitivity to methane for methane measurement and detection.

In addition other reactive or receptive biomolecules can target particular species, such as direct or indirect antibody reactions. For example, the probe may be a commercially available anti-HIV antibody which is reactive to target components of the HIV virus in a patient sample. Alternatively the probe may be a commercially available HIV antigen component, reactive to target human anti-HIV antibodies (from patient sample).

One of ordinary skill in the art can readily identify useful known probe-target combinations of biomolecules, such as enzymes and their substrates, antibodies and their specific antigens, and the like, which may be used to produce the molecular transducers for alternative embodiments, without departing from the spirit of the invention and without undue experimentation.

Note that the sensor arrays described above may be included in system embodiments sensitive to multiple targets. Alternatively, the arrays may providing multiple differently functionalized sensors for the same target species, to enhance selectivity, sensitivity, dynamic range, and the like.

### EXAMPLE B

### Sensor with Solution Deposited Nanotube Network

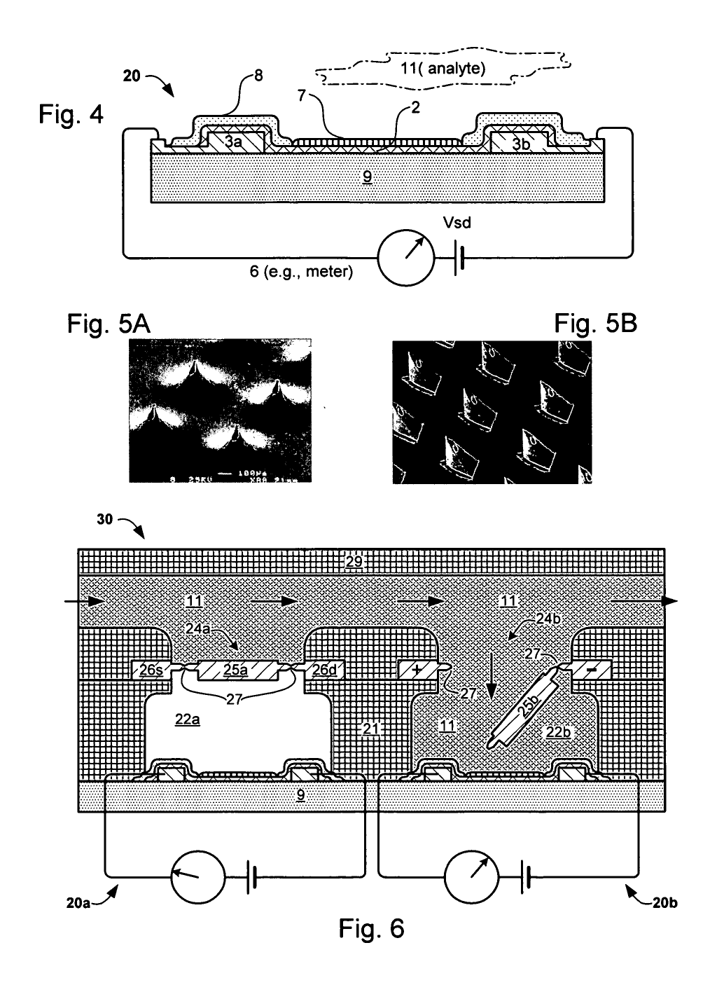

FIG. 4 is a diagram of an alternative examplary embodiment of a nanosensor 20 having aspects to the invention, including a network of carbon nanotubes. Certain elements are generally similar to those of FIG. 2A, and the same reference numbers are indicated.

Sensor 20 comprises a substrate 9, which preferably comprises a flexible sheet-like material such a polyester polymer (e.g., PET sheet). One or more electrodes (3a and 3b are shown) are arranged on the substrate. The electrode may comprise a metal, or may be formed from a paste or ink-like composition, such as carbon, graphite, conductive polymer, metalic ink compositions, and the like.

A nanostructure layer 2 (in this example a film including SWNTs) is deposited contacting the electrodes 3a (and 3b in this example). An optional functionalization or recognition layer 7 may be included in association with the layer 2. An additional passivation, protective or inhibiting layer 8 may cover electrodes 3 and all or a portion of layers 2 and 7.

In a preferred embodiment, substrate 9 is a flexible sheet having pre-patterned printed electrodes 3, permitting simplicity and cost reduction. Preferably the nanostructure layer 2 is formed by spraying or otherwise coating the patterned substrate with an liquid suspension of nanotubes. For example, SWNTs or MWNTs may be conveniently dispersed in aqueous suspension at a desired concentration, particularly where functionalization treatment of the SWNTs assist in making the nanotubes hydrophilic (see EXAMPLE D below).

In certain embodiments, recognition or detection material is deposited, reacted or bound to the nanotubes (or alternative nanostructures) prior to deposition of layer 2. Depending on the selected detection chemistry and analyte target, such pre-functionalization may eliminate the need for any distinct recognition layer 7.

Alternative nanotube dispersion techniques may also be employed, see for example, U.S. patent application Ser. No. 10/846,072 entitled “Flexible Nanotube Transistors”; and L. Hu et al., Percolation in Transparent and Conducting Carbon Nanotube Networks, Nano Letters (2004), 4, 12, 2513-17, each of which application and publication is incorporated herein by reference.

The nanostructure layer 2 may be deposited stepwise, with intermediate drying, to permit the density and conductivity of the layer 2 to be accurately controlled, such as by probe-testing the layer resistance or conductance between deposition steps, until a selected layer conductivity or resistance is achieved.

Suitable measurement circuitry is included in communication with electrodes 3a and 3b (and any optional additional electrodes, see EXAMPLE A above), here represented by meter 6 and source-drain power source Vsd.

### EXAMPLE C

### Sensor Integrated with Sampler Device

Sampling Components

In a medical application embodiment of the sensor provision is made for access to a bodily fluid. Most current prior art test strips use capillary blood, and it has been shown that interstitial fluid glucose concentrations are closely correlated with capillary blood. Electrochemical blood glucose measurement systems (including test strips and monitors) employ glucose oxidase enzyme (GOx) as an active detector substance, producing a current between spaced-apart electrodes upon reaction with glucose in a blood sample, the current providing a measurement signal upon insertion of the test strip into the associated monitor of the measurement system. See, for example, the FreeStyle™ Blood Glucose Monitoring System made by Abbott Laboratories of Abbott Park, Ill.

Certain preferred glucose sensor system embodiments having aspects of the invention include known components configured to sample blood or interstitial fluid, such as MEMS based “syringes” or needle arrays. FIG. 5A is a photomicrograph showing an array of pointed hollow out-of-plane silicon needles having a height of 200 microns and a channel diameter of 40 microns. FIG. 5B shows a generally comparable array of sampling/injecting micro-needles, available from NanoPass Ltd. of Haifa, Israel and Silex Microsystems AB of Järfälla, Sweden. Such sampling mechanisms may be integrated with sensors for direct sampling diagnostics, such as with the glucose sensor embodiments having aspects of the invention. Certain preferred glucose sensor system embodiments having aspects of the invention include known components which sequentially address compartments that can be attached to these needles.

FIG. 6 shows a multi-welled sample chip assembly 30, including a fusable port assembly 24 which permits remote electronic control of a well opening. A sample 11 may be introduced into assembly 30 by a number of alternative means, such as by connection to needle arrays such as shown in FIGS. 5A-B. One or more wells 22 are included in housing 21, in this example two are shown, 22a and 22b. A sensor device and associated measurement circuitry, e.g. such as shown in FIG. 2A and/or FIG. 4, is included in communication with each corresponding well, in this example, sensors 20a and 20b respectively. For convenience and cost control, multiple sensors may be formed on a common substrate 9 as shown.

Shell or wall 29 provides a fluid conduit to bring sample 11 adjacent to openings 24a and 24b in wells 22a and 22b, respectively. In this exemplary embodiment, each opening comprises a port 25 suspended between electrical leads 26s and 26d by means of fusible links 27. As shown for well 22b, the controlled application of electrical current (circuitry not shown) across leads 26s-26d causes one or more of links 27 to fuse or fracture, permitting the port 25b to release and/or rotate, to emit sample 11 to well 22b, so as to permit analyte detection by sensor 20b.

Alternative or addition known flow controls and/or valving may be included to regulate the emission of sample 11 to sensors of system 30, such as flexible pinch valves, piezoelectric actuators, and the like.

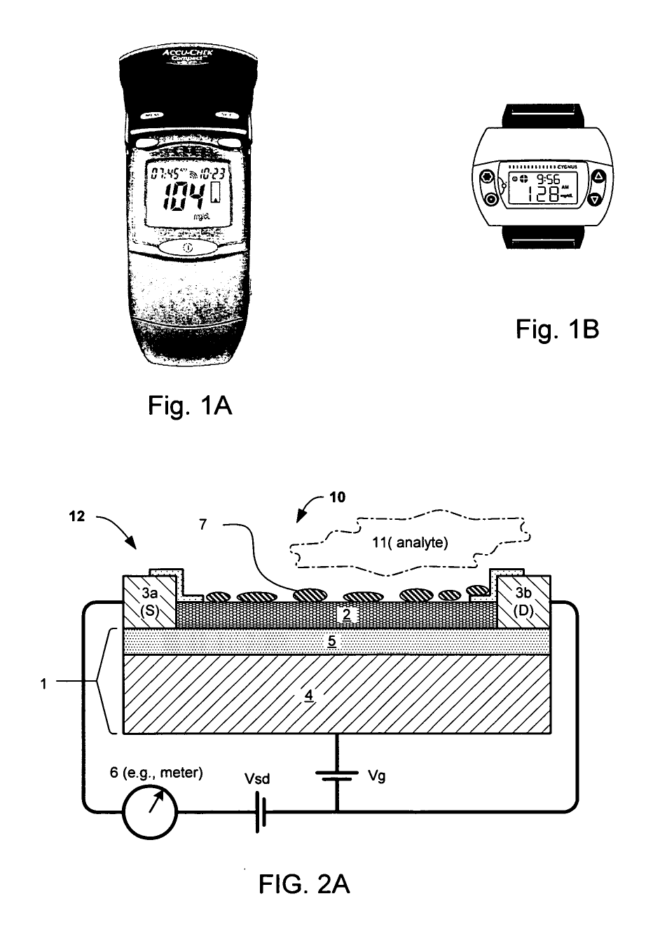

Certain preferred glucose sensor system embodiments are configured as a wearable sensor, similar in appearance to that shown in FIG. 1B. In addition to glucose sensor monitoring, sampling and/or measuring system embodiments, another aspect of the invention provides an integrated drug delivery embodiment, which may employ the components similar to the needles and/or sequentially addressable compartment components deliver and apply drug solutions, such as insulin.

Embodiments may include combinations of the described sample techniques with a sensor array in both disposable and reusable examples. The combination of MEMS sampling technology with highly sensitive nanobased detectors can provide a powerful solution for glucose monitoring. The scalability of the sensor platform is well suited for the high volume sensor use and cost analysis show a clear competitive and patient advantages.

### EXAMPLE D

### Printed Substrate Sensor Device

An alternative exemplary embodiment of a glucose sensor having aspects to the invention include single walled carbon nanotubes (SWNTs).

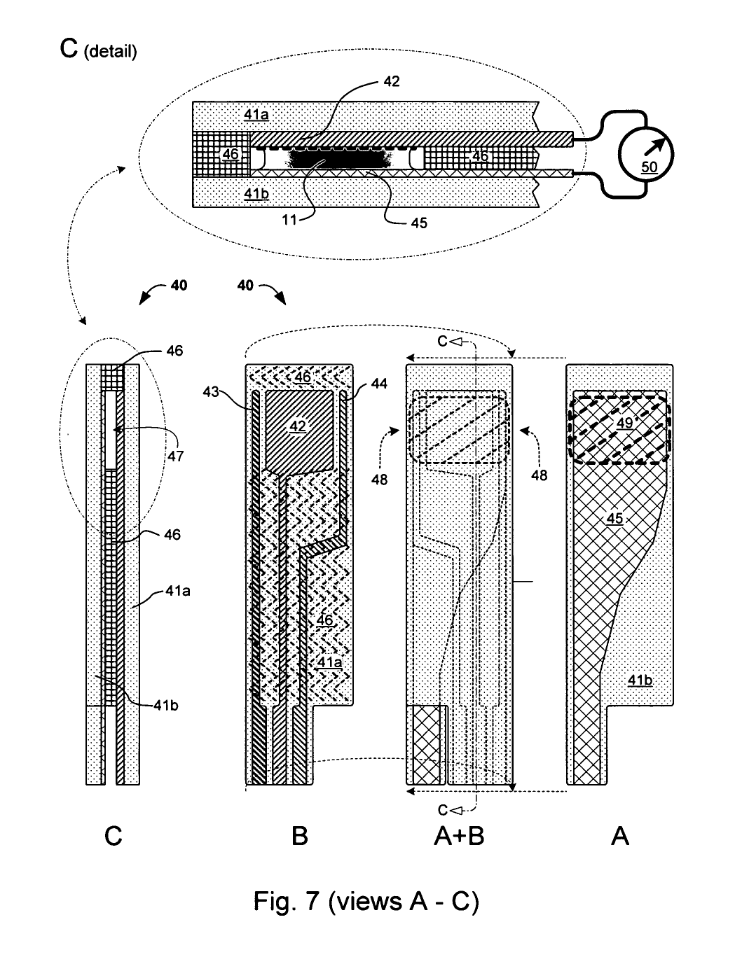

FIG. 7, Views A-C illustrate an exemplary electrochemical sensor 40, in this example configured as a blood test strip. A first substrate 41a (View A) and a second substrate 41b (View B) comprise a flexible sheet material such as PET polymer. A counter electrode 42 (preferably comprising a conductive ink) is printed, screen printed, shadow masked, or otherwise deposited on substrate 41a. Additional optional electrodes such reference electrode 43 and/or calibration electrode 44 may be deposited adjacent counter electrode 42. A conductive nanostructured film electrode 45 is deposited on substrate 41b. Film 45 may be printed, or may be spray deposited in the manner described with respect to the sensor 20 of FIG. 4.

Substrates 41a and 41b are preferably shaped so that they may be counter-posed and attached to one-another, such as by adhesive layer 46 to form a multilayer assembly (View A+B). Adhesive layer 46 may serve as an insulator to electrically isolate the counter electrode 42 (and also 43-44) from nanotube film 45 in the assembly, and the adhesive may also serve as a space to maintain a space between the substrate layers 41a-41b (best seen in cross-section View C). A gap or space in the adhesive layer 46 adjacent one end or other portion of the substrates 41 may serve to create sample well 47, comprising a void between the layers, the well 47 communicating with one or more sample ports 49 (in this example, ports 48a, 48b in the sides of sensor strip 40). Additional functionalization material 49 (e.g., comprising GOx for an exemplary glucose detector) may be deposited on either or both of electrode 42 and/or film 45 in line with sample well 47.

As seen in FIG. 7, View C (detail), a blood sample 11 may be drawn by capillary action into sample well 47 so as to contact both counter electrode 42 and film 45, and so as to dissolve associated functionalization 49. A signal (e.g., an electrochemically generated current) is measured by monitor circuitry 50 (diagramatically indicated by a meter), so as to produce a measurement of the glucose concentration (or other target analyte) in sample 11.

Advantageously, the nanotube film 45 may be pre-functionalized prior to deposition with nanoscale Pt particles. For example, surface oxidized SWNTs may be suspended (e.g., with sonification) in a solvent such as ethylene glycol-water mixture, containing a selected concentration of hexachloroplatinic acid. Preferably, the pH is adjusted (e.g. with NaOH) to about 13. The solution may be heated to about 140 deg. C in an oxygen-free atmosphere for an period to permit Pt reduction. The treated SWNTs may be centrifuged to remove solvent, and re-suspended in a desired deposition solvent prior to applying to substrate 41b. The concentrations of reagents and treatment temperature and time may be adjusted to produce the desired Pt content in the final film.

The conductive nanostructured film electrode 45 preferably comprises a film of carbon nanotubes, and more preferably comprises a highly-uniform network of SWNTs. In comparison to conventional glucose test strips (e.g., the FreeStyle™ system, and others) employing other conductive materials, the film 45 is configured to provide at least the following advantages:
(a) Accelerated response of sensor 40 to sample 11—film 45 provides a faster electrochemical response signal to reaction products (e.g., hydrogen peroxide and gluconic acid) from the enzymatic reaction to the glucose substrate.
(b) Film 45 provides a smooth, consistent surface for binding GOx (or other catalysts or cofactors), so as to produce a test strip with more consistent response to samples, so as to greatly reduce system calibration problems, leading to reduced costs, improved reliability and greater convenience of use.

The inclusion of platinum or Pt or other metal catalyst in the conductive nanostructured film electrode 45 are preferably nanoscale particles of a size generally on the order of the diameter of the nanotubes or smaller. In comparison to a film 45 without Pt functionalization, the Pt containing film provides at least the following advantages:
(a) The Pt nanoparticles provide an even faster electrochemical response signal to reaction products (e.g., hydrogen peroxide and gluconic acid).
(b) The Pt nanoparticles provide an even better binding point for immobilizing GOx (or other catalysts or cofactors), so as to advantageously produce a test strip with more consistent response to samples.
(c) The described process for pre-functionalizing the nanotubes with Pt (or other metal catalyst) permits convenient fabrication and a much more advantageous control of Pt particle size, distribution and content than other methods of applying or depositing Pt to a previously formed nanotube network, thus improving the value of (a) and (b) above.

### EXAMPLE E

### SWNT-PABS Functionalization

In this exemplary embodiment, the nanotubes are treated with a polymeric functionalization material. In this novel embodiment, the functionalization material includes poly (m-aminobenzene sulfonic acid) or PABS covalently attached to SWNTs (SWNT-PABS). The functionalized nanotubes may be included in any of the suitable sensor embodiments having aspects of the invention, such as the sensors described and shown in FIG. 2A, FIG. 4, and FIG. 7. In this example, the PABS-functionalized nanotubes were included in an electrochemical test strip generally similar to that shown in FIG. 7.

A composition of SWNT-PABS powder is commercially available from Carbon Solutions, Inc. of Riverside Calif., and may be made as described in B Zhao et al, “Synthesis and Properties of a Water-Soluble Single-Walled Carbon Nanotube-Poly(m-aminobenzene sulfonic acid) Graft Copolymer”, Adv Funct Mater (2004) Vol 14, No 1 pp 71-76, which article is incorporated by reference. An aqueous solution of SWNT-PABS may be prepared by ultrasonication (e.g., 1 mg/mL). After brief sonication, a homogeneous dispersion of carbon nanotubes was obtained.

The sensor in this example includes a flexible substrate comprising PET sheet (which are commercially available from McMaster-Carr Supply Company of Chicago Ill.). The carbon nanotubes dispersion was sprayed with an air brush in several steps with intermediate drying until the desired resistance was obtained. In this example, the deposition was carried out on with the substrate on a hot-plate with the temperature of about 75 degree C., and the dispersion was deposited step-wise until the half-inch resistance obtained using the pin probe was about 15 K Ohm.

The response of the sensors to glucose was demonstrated using the H2O2 solution as a simulant to glucose (note that the reaction of GOx with blood glucose produces peroxide, which in turn generates the measurement current). The 2.5 mM H2O2 solution was prepared corresponding to the 400 mg/dl of glucose concentration. A meter from Hypogard was used to record the reading of the glucose. The meter was calibrated based on the conductivity of CNT film.

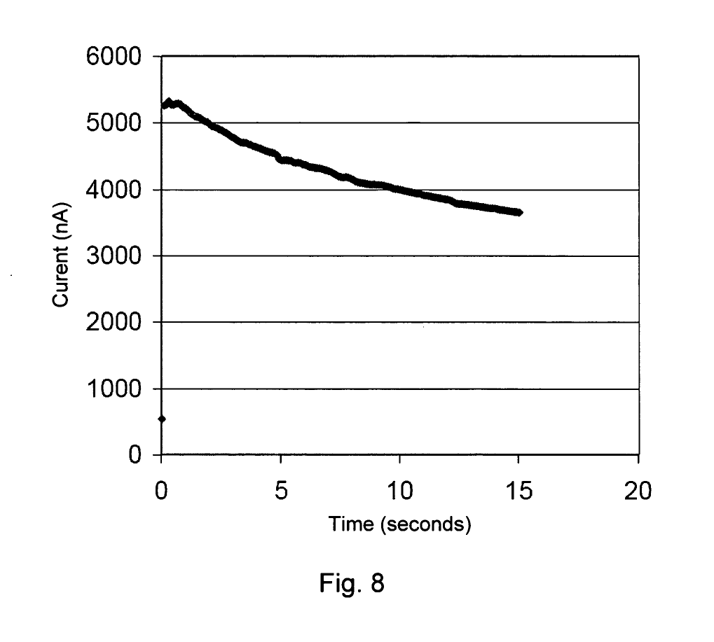

The response of PABS-SWNT strip sensor to 400 mg/dl glucose is as shown in FIG. 8. The meter records 367 mg/dl giving less than 10% error in the measurement of actual glucose concentration. Also the time (<2 seconds) required for measurement is less than in a conventional test strip, demostrating that the exemplary sensor is a faster sensor for glucose detection.

The molecular sensing mechanism of glucose for the SWNT-PABS can be understood considering the chemistry of polyaniline (PANI). PANIs are appealing for sensor applications because their electronic properties can be reversibly controlled by doping/dedoping at room temperature. The chemical modification of SWNTs significantly affected the sensitivity and reversibility of the behavior of the sensors. PABS is a water-soluble conducting polymer. The presence of SO3H groups improved the solubility and processability of this sulfonated polyaniline derivative, and it is especially attractive for introducing acid-base sensitivity together with a further doping response into sensor devices.

In this concept, the conducting polymer (PABS) acts as the immobilization matrix as well the physio-chemical transducer to convert a chemical signal (change of chemical potential of the microenvironment) into an electrical signal. The conducting polymer acts as the electron mediator while the carbon nanotubes provide enhanced surface area.

The sensors in this example may be made from pre-functionalized nanotubes, thus eliminating an additional step to functionalize nanotubes with polymers. Unlike conventional glucose biosensors, no electrochemical deposition is required in this case making it easy-to-fabricate sensor process.

Alternative techniques may be employed to functionalize nanotubes with other suitable conductive polymeric materials, such as PANI or may be employed. See, for example, the electrochemical treatments described in T Zhang et al, “Nanonose: Electrochemically Functionalized Single-Walled Carbon Nanotube Gas Sensor Array”, Proc. 208th Meeting of Electrochemical Society (Los Angeles, Calif. Oct. 16-21, 2005), which is incorporated by reference.

The SWNTs (or alternative nanostructures) functionalized with PABS (other alternative conductive polymers such as other polyaniline derivatives) are advantageously employed to comprise the nanostructured film electrode 45 as shown in FIG. 7. In comparison to conventional glucose test strips (e.g., the FreeStyle™ system, and others) employing other conductive materials, the film 45 comprising is configured to provide at least the following advantages:
(a) The example electrode film 45 having conductive polymer functionalization (e.g., PABS) provides a faster electrochemical response signal to reaction products (e.g., hydrogen peroxide and gluconic acid) from the enzymatic reaction to the glucose substrate. In addition, where, as in this example, there is a covalent bond between the conductive polymer (e.g., PABS) and the conductive nanostructure (e.g., SWNTs), the accelerated response is particularly notable.
(b) The example electrode film 45 having conductive polymer functionalization provides a smooth, consistent surface for binding GOx (or other catalysts or cofactors), providing the advantageous described above with respect to Pt functionalized nanotube films.
(c) In many cases, the example electrode film having conductive polymer functionalization of this EXAMPLE E provides better sensor properties and response, in comparison to the alternative film having Pt functionalization as described in EXAMPLE D.
(d) An additional alternative electrode film 45 may advantageously have conductive polymer functionalization in combination with Pt functionalization. For example, SWNTs (or other nanostructures) may be pre-functionalized with both PABS and Pt, and then deposited as a film electrode. In yet other alternative examples, a film electrode may be deposited from a mixture of differently-functionalized nanotubes (e.g. SWNT/PABS+SWNT/Pt); or a film electrode may be deposited in layers (e.g., stepwise deposition) of differently-functionalized nanotubes (e.g. SWNT/PABS layered with SWNT/Pt).

Having thus described a preferred embodiment of the nanotube sensor device, it should be apparent to those skilled in the art that certain advantages of the within system have been achieved. It should also be appreciated that various modifications, adaptations, and alternative embodiments thereof may be made within the scope and spirit of the present invention.

1. A nanoelectronic sensor for analyte detection, comprising:
a substrate;
at least one electrical contact disposed adjacent the substrate;
a conductive layer disposed on the substrate, the conductive layer comprising a plurality of nanostructured material;
the conductive layer including a conductive polymeric material in association with the the conductive layer;
electrical measurement circuitry in communication with the at least one contact and in communication with the conductive layer,
the conductive layer disposed so as to permit contact of the conductive layer with an analyte sample so as to transmit at least one signal to the electrical measurement circuitry in response to the sample;
the electrical measurement circuitry configured to detect a concentration of a selected analyte in the sample using the at least one signal.

2. A nanoelectronic sensor as in claim 1, wherein the contact of the conductive layer with an analyte sample is arranged so as to be in association with a quantity of glucose oxidase enzyme, the at least one signal being in response to the contact of glucose oxidase with the sample, and wherein the electrical measurement circuitry measures a concentration of glucose in the sample using the at least one signal.

3. A nanoelectronic sensor as in claim 1, wherein the nanostructured material is selected from the group consisting of single-walled carbon nanotubes, multiwalled carbon nanotubes, and nanowires.

4. A nanoelectronic sensor as in claim 1, wherein the conductive polymeric material comprises a polyaniline derivative.

5. A nanoelectronic sensor as in claim 4, wherein the polyaniline derivative comprises poly (m-aminobenzene sulfonic acid).

6. A nanoelectronic sensor as in claim 1, wherein the conductive layer is formed by application of a suspension of nanotubes in a solvent to the substrate.

7. A nanoelectronic sensor as in claim 6, wherein nanotubes are treated to bind to the conductive polymeric material prior to the application of a suspension of nanotubes in a solvent to the substrate.

8. A nanoelectronic sensor as in claim 1, wherein the conductive layer further comprises nanoparticles, the nanoparticles comprising a transition metal.

9. A nanoelectronic sensor as in claim 8, wherein the transition metal comprises platinum.

10. A nanoelectronic sensor system for analyte detection, comprising:
a substrate;
a nanostructured layer comprising a plurality of nanostructured material disposed adjacent the substrate, and including a conductive polymeric material in association with the nanostructured material;
at least one electrical contact disposed adjacent the substrate in electrical communication with the nanostructured layer;
electrical measurement circuitry in communication with the at least one contact;
the nanostructured layer configured to transmit at least one signal to the electrical measurement circuitry in response to a target analyte;
the electrical measurement circuitry configured to detect a target analyte using the at least one signal.

11. A nanoelectronic sensor system as in claim 10, wherein the nanostructured material is selected from the group consisting of single-walled carbon nanotubes, multiwalled carbon nanotubes, and nanowires.

12. A nanoelectronic sensor system as in claim 10, wherein the conductive polymeric material comprises a polyaniline derivative.

13. A nanoelectronic sensor system as in claim 12, wherein the polyaniline derivative comprises poly (m-aminobenzene sulfonic acid).

14. A nanoelectronic sensor system as in claim 10, wherein the nanostructured layer is formed application of a suspension of nanotubes in a solvent to the substrate.

15. A nanoelectronic sensor system as in claim 14, wherein nanotubes are treated to bind to the conductive polymeric material prior to the application of a suspension of nanotubes in a solvent to the substrate.

16. A nanoelectronic sensor system as in claim 10, wherein the at least one electrical contact comprises a spaced-apart pair of electrical contacts in electrical communication with the nanostructured layer, the pair of electrical contacts in communication with the electrical measurement circuitry and configured to produce a signal indicative of an electrical property of the nanostructured layer in response to a target analyte.

17. A nanoelectronic sensor system as in claim 16, further comprising a gate electrode disposed in capacitive association with the nanostructured layer and in communication with the electrical measurement circuitry.

18. A nanoelectronic sensor system as in claim 10, further comprising a counter electrode disposed in spaced apart association with the nanostructured layer, the counter electrode in communication with the electrical measurement circuitry.

19. A nanoelectronic sensor system as in claim 18, wherein the electrical measurement circuitry is configured to measure a capacitance property of the nanostructured layer in response to the target analyte.

20. A nanoelectronic sensor system as in claim 18, wherein the electrical measurement circuitry is configured to measure an electrochemical reaction in association with the nanostructured layer in response to the target analyte.

21. A nanoelectronic sensor system as in claim 18, wherein the electrical measurement circuitry is configured to measure a electron emission breakdown voltage of the nanostructured layer in response to the target analyte.
